(19) United States
(12) Patent Application Publication
(10) Pub. No.: US 2025/0359775 A1
(43) Pub. Date: Nov. 27, 2025

(54) SCOLIOSIS RECOGNITION SYSTEM AND METHOD BASED ON ACTIVE MILLIMETER WAVE IMAGING TECHNOLOGY

(71) Applicant: Anhui Yuanshuo Terahertz Technology Co., LTD, Hefei (CN)

(72) Inventor: Zhenli ZHAO, Hefei (CN)

(73) Assignee: Anhui Yuanshuo Terahertz Technology Co., LTD, Hefei (CN)

(21) Appl. No.: 19/030,685

(22) Filed: Jan. 17, 2025

Related U.S. Application Data
(63) Continuation of application No. PCT/CN2024/126638, filed on Oct. 23, 2024.

(30) Foreign Application Priority Data
May 23, 2024 (CN) 202410648978.2

(51) Int. Cl. A61B 5/107

(52) U.S. Cl. CPC A61B 5/1071; A61B 5/1079

(57) ABSTRACT

The present invention relates to scoliosis recognition, and in particular to a scoliosis recognition system and method based on active millimeter wave imaging technology. An active millimeter wave scanning assembly is configured to transmit millimeter wave signals of a specific frequency band to the back of a human body, receive echo signals of the back of the human body, and send the received echo signals to a terminal control device to realize non-invasive scanning of the back of the human body. An active millimeter wave flat panel device is configured to, under the control of a terminal control device, drive the active millimeter wave scanning assembly to scan in a sequence from top to bottom so as to obtain 3D morphological data of the back of the human body. A pressure sensing device is configured to measure pressure distribution data when the human body stands.

### CROSS-REFERENCE TO RELATED APPLICATIONS

The application claims priority to Chinese patent application No. 202410648978.2, filed on May 23, 2024, the entire contents of which are incorporated herein by reference.

### TECHNICAL FIELD

The present invention relates to scoliosis recognition, and more particularly to a scoliosis recognition system and method based on active millimeter wave imaging technology.

### BACKGROUND

In the medical field, idiopathic scoliosis is a common disease among adolescents, and its severity cannot be ignored. The prominent feature of this condition is the abnormal curvature of the spine in a frontal view, which not only affects the patient's physical appearance but may also cause back pain, difficulty breathing, and other serious complications. Therefore, timely detection and treatment of scoliosis is essential to guarantee the health of patients and improve their quality of life.

Although significant progress has been made in the diagnosis and treatment of scoliosis, in clinical practice, the symptoms of mild to moderate scoliosis are often overlooked by patients and their parents due to their difficulty in being detected. In addition, schools and medical personnel have insufficient understanding of this disease, and missed diagnoses often occur, which undoubtedly increases the risk of worsening the condition.

Currently, medical imaging tests, such as X-rays and CT scans, play a key role in the diagnosis of scoliosis. However, the above-mentioned detection methods inevitably cause a certain degree of ionizing radiation damage to patients, especially to children and adolescents in the growth and development stage. In addition, medical imaging techniques are not as suitable as conventional monitoring tools due to their cost of use and operational complexity.

Although B-ultrasound has certain application value in detecting scoliosis, it is rarely used in actual daily monitoring, mainly due to the lack of specialized physicians to operate and interpret the test results, which limits the application of B-ultrasound technology in large-scale screening and daily monitoring.

As a widely praised screening method, the method of anteflexion measurement with a horizontal ruler is popular because of its convenient detection. However, this method has low accuracy and is easily affected by factors such as human body shaking, leading to errors in the detection results. Furthermore, the method is labor-dependent and time-consuming.

As a non-invasive detection method, binocular vision inspection can reduce the dependence on manpower to a certain extent, but it also has some limitations, such as being easily affected by external factors such as changes in ambient light and differences in detection angles. Similar to anteflexion measurement with a horizontal ruler, binocular vision detection is also susceptible to human body jitter and interference, which limits its application in daily monitoring.

In summary, the commonly used methods for detecting scoliosis currently have certain limitations, especially when faced with the demand for high-throughput and rapid periodic screening, the shortcomings of these methods become particularly evident. Therefore, it is urgent to develop new scoliosis detection technology to improve the accuracy and efficiency of scoliosis recognition.

### SUMMARY

### The Technical Problem to be Solved

In view of the above-mentioned shortcomings of the prior art, the present invention provides a scoliosis recognition system and method based on active millimeter wave imaging technology, which can effectively overcome the shortcomings of the prior art, such as low accuracy and efficiency of scoliosis recognition in the face of high-throughput and rapid periodic screening.

### (II) Technical Solutions

To achieve the above objectives, the present invention is implemented through the following technical solution:
a scoliosis recognition system based on active millimeter wave imaging technology, including an active millimeter wave scanning assembly, an active millimeter wave flat panel device, a pressure sensing device, a plantar indication module, a terminal display device, and a terminal control device.

The active millimeter wave scanning assembly is configured to transmit a millimeter wave signal of a specific frequency band to the back of a human body, receive an echo signal of the back of the human body, and send the received echo signal to the terminal control device so as to realize non-invasive scanning on the back of the human body.

The active millimeter wave flat panel device is configured to, under the control of the terminal control device, drive the active millimeter wave scanning assembly to scan in a sequence from top to bottom so as to obtain 3D morphological data of the back of the human body.

The pressure sensing device is configured to measure pressure distribution data when the human body stands, and send the collected pressure distribution data to the terminal control device.

The terminal control device is configured to evaluate whether the standing posture and standing position of the human body are standard based on the pressure distribution data, generate corresponding feedback information when the standing posture and standing position are not standard, and instruct the user to adjust the standing posture and standing position via the plantar indication module, and at the same time, detect whether there is scoliosis based on the echo signal, generate a corresponding diagnosis report according to the scoliosis recognition result, and send the scoliosis recognition result and the diagnosis report together to the terminal display device.

The terminal display device is configured to display the scan progress, device status, scoliosis recognition result, and diagnosis report in real time.

Preferably, the terminal control device includes a pressure distribution data pre-processing module, a pressure distribution data analysis module, a key position detection module, and a standing posture and standing position evaluation module.

The pressure distribution data pre-processing module is configured to perform digital processing on the received pressure distribution data, filter noise, and convert the same into a data format suitable for algorithm analysis through normalization processing;
The pressure distribution data analysis module is configured to perform a two-foot pressure distribution analysis based on the pre-processed pressure distribution data and determine the center of gravity weight of the human body and the pressure distribution symmetry.

The key position detection module is configured to detect the positions of the heel, the arch, and the forefoot of the human body based on the pre-processed pressure distribution data, and perform standing position comparison and analysis with a foot-type standing position mark on the pressure sensing device.

The standing posture and standing position evaluation module is configured to evaluate whether the standing posture and standing position of the human body are standard by combining the pressure distribution symmetry and the standing position comparison and analysis result, generate corresponding feedback information when the standing posture and standing position are not standard, and instruct the user to adjust the standing posture and standing position by the plantar indication module by sending the feedback information to the plantar indication module.

The plantar indication module is configured to instruct the user to adjust the standing posture and standing position through a voice or visual prompt.

Preferably, the pressure distribution data analysis module is configured to perform a two-foot pressure distribution analysis based on the pre-processed pressure distribution data and determine the center of gravity weight of the human body and the pressure distribution symmetry, including:
calculating the two-foot pressure distribution using the following formula:

$$\begin{matrix}{C}_{L} ( t ) = \frac{{{\sum }^{m}}_{i = 1} [ {P}_{i} ( t ) {x}_{i} ]}{{{\sum }^{m}}_{i = 1} {P}_{i} ( t )} ; \\ {C}_{R} ( t ) = \frac{{{\sum }^{n}}_{j = 1} [ {P}_{j} ( t ) {y}_{j} ]}{{{\sum }^{n}}_{j = 1} {P}_{j} ( t )} ;\end{matrix}$$

where CL(t) is the left foot pressure value distribution at time t, Pi(t) is the pressure value detected by the ith pressure sensor below the left foot at time t; xi is the coordinate of the ith pressure sensor below the left foot on the plane; and m is the number of pressure sensors below the left foot;
CR(t) is the pressure value distribution of the right foot at time t; Pj(t) is the pressure value detected by the jth pressure sensor below the right foot at time t; yj is the coordinate of the jth pressure sensor below the right foot on the plane; and nis the number of pressure sensors below the right foot;
calculating the center of gravity weight of the human body based on the two-foot pressure distribution using the following formula:

$$\begin{matrix}{W}_{L} ( t ) = \frac{{C}_{L} ( t ) {S}_{L} + {C}_{R} ( t ) {S}_{L}}{{S}_{L} + {S}_{R}} ; \\ {W}_{R} ( t ) = \frac{{C}_{L} ( t ) {S}_{R} + {C}_{R} ( t ) {S}_{R}}{{S}_{L} + {S}_{R}} ;\end{matrix}$$

where WL(t) and WR(t) are respectively the center of gravity weight of the left foot and the center of gravity weight of the right foot at time t; and SL and SR are respectively the contact area of the left foot and the contact area of the right foot;
based on the center of gravity weight of the human body, calculating a pressure distribution symmetry score using the following formula:

$$S ( t ) = 1 - \frac{\vert {W}_{L} ( t ) - {W}_{R} ( t ) \vert}{max [ {W}_{L} ( t ) , {W}_{R} ( t ) ]} ;$$

where S(t) is the pressure distribution symmetry score at time t, and when the pressure distribution symmetry score is higher than 0.85, it indicates that the standing posture of the human body is symmetrical and meets the requirements of posture symmetry; otherwise, the standing posture of the human body is asymmetrical and does not meet the requirements of posture symmetry.

Preferably, the terminal control device includes an echo signal pre-processing module, a back key point recognition module, a spine fitting analysis module, and a diagnosis report generation module.

The echo signal pre-processing module is configured to filter and denoise the received echo signal, and convert the same into 3D morphological data of the back of the human body;
The back key point recognition module is configured to recognize the back key points from the 3D morphological data of the back of the human body using the depth learning algorithm BlazePose, and then segment the 3D data of the back of the human body;
The spine fitting analysis module is configured to, based on the 3D data of the back of the human body, recognize a preliminary curve of a spine contour using an improved active contour algorithm, and then perform a cubic B-spline curve smoothing to obtain a fitting curve of the spine contour;
The diagnosis report generation module is configured to detect whether there is scoliosis in the human body based on the calculated Cobb angle of the spine, generate a corresponding diagnosis report according to the scoliosis recognition result, and send the scoliosis recognition result and the diagnosis report together to the terminal display device.

The back key points include the bilateral acromion, scapula, back dimples, and iliac crest, and the diagnosis report includes the Cobb angle of the spine, the severity range of scoliosis, and the corresponding medical suggestions.

A scoliosis recognition method based on active millimeter wave imaging technology, including the steps of:
S1, starting the active millimeter wave flat panel device and the active millimeter wave scanning assembly, allowing the system to enter a state to be scanned, and automatically executing a checking and spatial calibration procedure so as to ensure scanning accuracy;
S2, allowing the user to stand in a designated position according to the foot-type standing position mark on the pressure sensing device;
S3, measuring, via the pressure sensing device, pressure distribution data when the human body stands, and sending the collected pressure distribution data to the terminal control device;
S4, evaluating, via the terminal control device, whether the standing posture and standing position of the human body are standard based on the pressure distribution data, generating corresponding feedback information when the standing posture and standing position are not standard, and instructing the user to adjust the standing posture and standing position via the plantar indication module;
S5, repeating S3-S4 until the standing posture and standing position meet the standard requirements;
S6, starting, via the active millimeter wave flat panel device and the active millimeter wave scanning assembly, to perform non-invasive scanning on the back of the human body; transmitting, via the active millimeter wave scanning assembly, a millimeter wave signal of a specific frequency band to the back of the human body, and sending the received echo signal of the back of the human body to the terminal control device;
S7, detecting, via the terminal control device, whether there is scoliosis in the human body based on the echo signal, generating a corresponding diagnosis report according to the scoliosis recognition result, and sending the scoliosis recognition result and the diagnosis report together to the terminal display device; and
S8, displaying, via the terminal display device, the scoliosis recognition result and the diagnosis report in real time, and performing, via the terminal control device, electronic transmission, or printing for the medical personnel to further analyze and formulate the treatment plan.

Preferably, in S4, evaluating, via the terminal control device, whether the standing posture and standing position of the human body are standard based on the pressure distribution data, generating corresponding feedback information when the standing posture and standing position are not standard, and instructing the user to adjust the standing posture and standing position via the plantar indication module, includes:
S41, performing digital processing on the received pressure distribution data, filtering noise, and converting the same into a data format suitable for algorithm analysis through normalization processing;
S42, performing a two-foot pressure distribution analysis based on the pre-processed pressure distribution data to determine the center of gravity weight of the human body and the pressure distribution symmetry;
S43, detecting the positions of the heel, the arch, and the forefoot of the human body based on the pre-processed pressure distribution data, and performing standing position comparison and analysis with a foot-type standing position mark on the pressure sensing device; and
S44, evaluating whether the standing posture and standing position of the human body are standard by combining the pressure distribution symmetry and the standing position comparison and analysis result, generating corresponding feedback information when the standing posture and standing position are not standard, and instructing the user to adjust the standing posture and standing position by the plantar indication module by sending the feedback information to the plantar indication module;
wherein the plantar indication module is configured to instruct the user to adjust the standing posture and standing position through a voice or visual prompt.

Preferably, in S7, detecting, via the terminal control device, whether there is scoliosis in the human body based on the echo signal, generating a corresponding diagnosis report according to the scoliosis recognition result, and sending the scoliosis recognition result and the diagnosis report together to the terminal display device, includes:
S71, filtering, and denoising the received echo signal, and converting the same into 3D morphological data of the back of the human body;
S72, recognizing the back key points from the 3D morphological data of the back of the human body using the depth learning algorithm BlazePose, and then segmenting the 3D data of the back of the human body;
S73, based on the 3D data of the back of the human body, recognizing a preliminary curve of a spine contour using an improved active contour algorithm, and then performing a cubic B-spline curve smoothing to obtain a fitting curve of the spine contour;
S74, detecting whether there is scoliosis in the human body based on the calculated Cobb angle of the spine, generating a corresponding diagnosis report according to the scoliosis recognition result, and sending the scoliosis recognition result and the diagnosis report together to the terminal display device;
wherein, the back key points include the bilateral acromion, scapula, back dimples, and iliac crest, and the diagnosis report includes the Cobb angle of the spine, the severity range of scoliosis, and the corresponding medical suggestions.

Preferably, in S73, based on the 3D data of the back of the human body, using an improved active contour algorithm to identify a preliminary curve of the spine contour, and then performing cubic B-spline curve smoothing to obtain a fitting curve of the spine contour, includes:
S731, initializing a curve at a central axis position of the 3D data on the back of the human body as an initial curve approximating a real spine contour;
S732, constructing by using the active contour algorithm an energy function including an internal energy term and an external energy term, wherein the internal energy maintains the smoothness and structure of the curve, and the external energy attracts the initial curve to the edge of the spine by using the gradient size based on the image gradient information;
S733, updating the initial curve position according to the gradient descending direction of the energy function, and iteratively minimizing the energy function so that the initial curve gradually approaches the real spine contour;
S734, setting a stop condition that the reduction amplitude of the energy function is lower than a threshold value or the number of iterations reaches an upper limit, and after the iteration is completed, eliminating abnormal points to obtain a preliminary curve of the spine contour; and
S735, obtaining the fitting curve of the spine contour using the cubic B-spline curve smoothing.

### (III) Advantageous Effects

Compared with the prior art, the scoliosis recognition system and method based on the active millimeter wave imaging technology provided by the present invention provides a non-radiation, high-accuracy, high-throughput, rapid periodicity, and user-friendly scoliosis recognition system and method by introducing the active millimeter wave imaging technology. Based on the millimeter wave radar principle, 3D morphological data of the back of the human body is obtained by transmitting and receiving millimeter wave signals, and then the depth learning algorithm and surface fitting algorithm are applied to respectively identify the key points of the back and analyze the 3D morphology of the spine. Finally, the Cobb angle of the spine is calculated and the corresponding diagnosis report is generated. This method not only improves the accuracy and efficiency of scoliosis recognition but also helps to reduce the risk of missed diagnosis and misdiagnosis. Meanwhile, it protects the privacy of users and lays a solid foundation for the early treatment of scoliosis patients and the improvement of quality of life.

The present invention specifically includes the following beneficial effects:
1) protecting user privacy: the use of active millimeter wave imaging technology for scoliosis detection does not require users to undress or adopt specific postures, thus protecting their privacy;
2) no direct contact required: millimeter wave imaging technology allows non-invasive scanning within a certain distance, without the need for direct physical contact between medical personnel and users, while respecting the privacy of users, it also reduces the workload of medical personnel;
3) convenient detection process: the whole scanning process is rapid and automatic, and the user only needs to stand on the scanning platform according to the instructions without other complex coordination, so the detection process is more convenient;
4) reducing human error: the adoption of automated scanning and analysis process reduces the dependence on the operator, so as to avoid the occurrence of inaccurate scoliosis recognition caused by human factors; and
5) immediate result feedback: the system provides real time scoliosis recognition results and diagnosis reports, does not require long waiting times, meeting the needs of high-throughput and rapid periodic screening.

### BRIEF DESCRIPTION OF DRAWINGS

In order to provide a clearer explanation of the embodiments of the present invention or the technical solutions in the prior art, a brief introduction will be given below to the accompanying drawings required in the description of the embodiments or prior art. It is obvious that the drawings in the description below are only some embodiments of the present invention, and it would be obvious for a person skilled in the art to obtain other drawings according to these drawings without involving any inventive effort.

FIG. 1 is a schematic diagram of the system of the present invention;

FIG. 2 is a top-view schematic diagram of a pressure sensing device in FIG. 1 of the present invention;

FIG. 3 is a schematic diagram of the recognition results of key points on the back of the present invention; and

FIG. 4 is a flow schematic diagram of the present invention.

### DETAILED DESCRIPTION OF THE EMBODIMENTS

In order to clarify the purpose, technical solution, and advantages of the embodiments of the present invention, the following will provide a clear and complete description of the technical solutions in the embodiments of the present invention in conjunction with the accompanying drawings of the embodiments. Obviously, the described embodiments are a portion of the embodiments of the present invention and not all of the embodiments. Based on the embodiments of the present invention, all Other embodiments obtained by a person of ordinary skill in the art without inventive effort fall within the scope of the present invention.

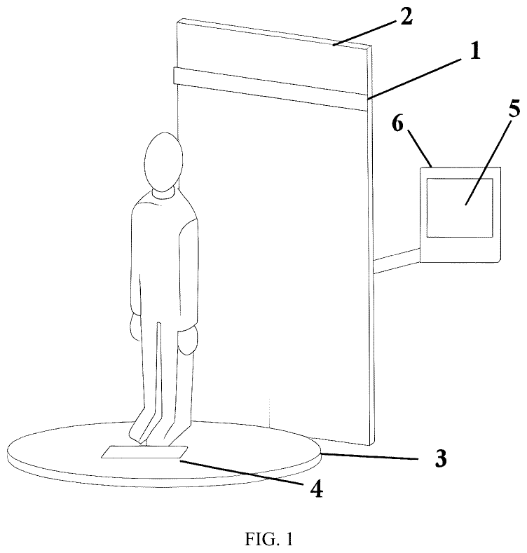

A scoliosis recognition system based on active millimeter wave imaging technology, as shown in FIG. 1, includes an active millimeter wave scanning assembly 1, an active millimeter wave flat panel device 2, a pressure sensing device 3, a plantar indication module 4, a terminal display device 5, and a terminal control device 6.

The active millimeter wave scanning assembly 1 is configured to transmit a millimeter wave signal of a specific frequency band (Ka-band) to the back of a human body, receive an echo signal of the back of the human body, and send the received echo signal to the terminal control device 6 so as to realize non-invasive scanning on the back of the human body.

The active millimeter wave flat panel device 2 is configured to, under the control of the terminal control device 6, drive the active millimeter wave scanning assembly 1 to scan in a sequence from top to bottom so as to obtain 3D morphological data of the back of the human body.

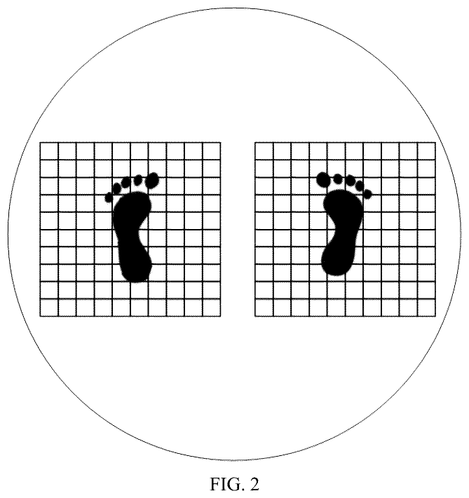

The pressure sensing device 3 is configured to measure pressure distribution data when the human body stands (as shown in FIG. 2, using two groups of pressure sensor arrays to respectively collect pressure distribution data of two feet), and send the collected pressure distribution data to the terminal control device 6.

The terminal control device 6 is configured to evaluate whether the standing posture and standing position of the human body are standard based on the pressure distribution data, generate corresponding feedback information when the standing posture and standing position are not standard, and instruct the user to adjust the standing posture and standing position via the plantar indication module 4, and at the same time, detect whether there is scoliosis based on the echo signal, generate a corresponding diagnosis report according to the scoliosis recognition result, and send the scoliosis recognition result and the diagnosis report together to the terminal display device 5.

The terminal display device 5 is configured to display the scan progress, device status, scoliosis recognition result, and diagnosis report in real time.
\textcircled{1} The terminal control device 6 includes a pressure distribution data pre-processing module, a pressure distribution data analysis module, a key position detection module, and a standing posture and standing position evaluation module.

The pressure distribution data pre-processing module is configured to perform digital processing on the received pressure distribution data, filter noise, and convert the same into a data format suitable for algorithm analysis through normalization processing (so as to eliminate the influence caused by different user weights).

The pressure distribution data analysis module is configured to perform a two-foot pressure distribution analysis based on the pre-processed pressure distribution data and determine the center of gravity weight of the human body and the pressure distribution symmetry.

The key position detection module is configured to detect the positions of the heel, the arch, and the forefoot of the human body based on the pre-processed pressure distribution data, and perform standing position comparison and analysis with a foot-type standing position mark on the pressure sensing device 3 (as shown in FIG. 2).

The standing posture and standing position evaluation module is configured to evaluate whether the standing posture and standing position of the human body are standard by combining the pressure distribution symmetry and the standing position comparison and analysis result, generate corresponding feedback information when the standing posture and standing position are not standard, and instruct the user to adjust the standing posture and standing position by the plantar indication module 4 by sending the feedback information to the plantar indication module 4.

The plantar indication module 4 instructs the user to adjust the standing posture and standing position through a voice or visual prompt (such as “Please move the left foot a little forward” or “Please straighten the waist”).

Specifically, the pressure distribution data analysis module is configured to perform a two-foot pressure distribution analysis based on the pre-processed pressure distribution data and determine the center of gravity weight of the human body and the pressure distribution symmetry, including:
calculating the two-foot pressure distribution using the following formula:

$$\begin{matrix}{C}_{L} ( t ) = \frac{{{\sum }^{m}}_{i = 1} [ {P}_{i} ( t ) {x}_{i} ]}{{{\sum }^{m}}_{i = 1} {P}_{i} ( t )} ; \\ {C}_{R} ( t ) = \frac{{{\sum }^{n}}_{j = 1} [ {P}_{j} ( t ) {y}_{j} ]}{{{\sum }^{n}}_{j = 1} {P}_{j} ( t )} ;\end{matrix}$$

where CL(t) is the left foot pressure value distribution at time t, Pi(t) is the pressure value detected by the ith pressure sensor below the left foot at time t, xi is the coordinate of the ith pressure sensor below the left foot on the plane; and m is the number of pressure sensors below the left foot;
C_{R}(t) is the pressure value distribution of the right foot at time t, Pⱼ(t) is the pressure value detected by the jth pressure sensor below the right foot at time t; yⱼ is the coordinate of the jth pressure sensor below the right foot on the plane; and n is the number of pressure sensors below the right foot;
calculating the center of gravity weight of the human body based on the two-foot pressure distribution using the following formula:

$$\begin{matrix}{W}_{L} ( t ) = \frac{{C}_{L} ( t ) {S}_{L} + {C}_{R} ( t ) {S}_{L}}{{S}_{L} + {S}_{R}} ; \\ {W}_{R} ( t ) = \frac{{C}_{L} ( t ) {S}_{R} + {C}_{R} ( t ) {S}_{R}}{{S}_{L} + {S}_{R}} ;\end{matrix}$$

where WL(t) and WR(t) are respectively the center of gravity weight of the left foot and the center of gravity weight of the right foot at time t; and SL and SR are respectively the contact area of the left foot and the contact area of the right foot;
based on the center of gravity weight of the human body, calculating a pressure distribution symmetry score using the following formula:

$$S ( t ) = 1 - \frac{\vert {W}_{L} ( t ) - {W}_{R} ( t ) \vert}{max [ {W}_{L} ( t ) , {W}_{R} ( t ) ]} ;$$

where S(t) is the pressure distribution symmetry score at time t, and when the pressure distribution symmetry score is higher than 0.85, it indicates that the standing posture of the human body is symmetrical and meets the requirements of posture symmetry; otherwise, the standing posture of the human body is asymmetrical and does not meet the requirements of posture symmetry.
\textcircled{2} The terminal control device 6 includes an echo signal pre-processing module, a back key point recognition module, a spine fitting analysis module, and a diagnosis report generation module.

The echo signal pre-processing module is configured to filter and denoise the received echo signal and convert the same into 3D morphological data of the back of the human body.

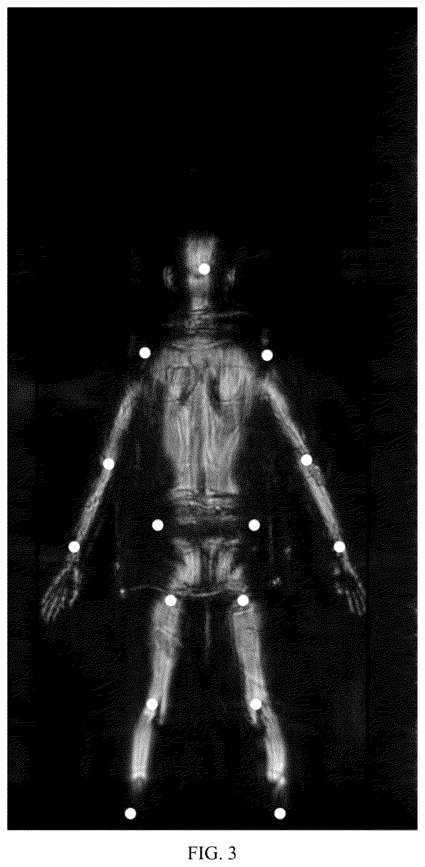

The back key point recognition module is configured to recognize the back key points from the 3D morphological data of the back of the human body (as shown in FIG. 3) using the depth learning algorithm BlazePose, and then segment the 3D data of the back of the human body.

The spine fitting analysis module is configured to, based on the 3D data of the back of the human body, recognize a preliminary curve of a spine contour using an improved active contour algorithm, and then perform a cubic B-spline curve smoothing to obtain a fitting curve of the spine contour.

The diagnosis report generation module is configured to detect whether there is scoliosis in the human body based on the calculated Cobb angle of the spine (an important parameter for measuring the degree of scoliosis), generate a corresponding diagnosis report according to the scoliosis recognition result, and send the scoliosis recognition result and the diagnosis report together to the terminal display device 5.

Wherein, the back key points include the bilateral acromion, scapula, back dimples, and iliac crest, and the diagnosis report includes the Cobb angle of the spine, the severity range of scoliosis, and the corresponding medical suggestions.

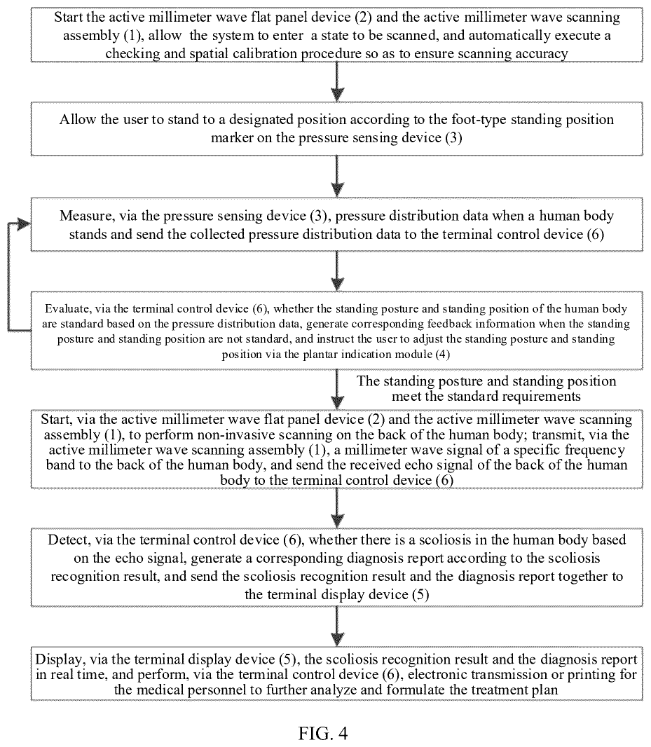

In the technical solution of the present application, also provided is a scoliosis recognition method based on active millimeter wave imaging technology, as shown in FIG. 4, including the following steps:
S1, starting the active millimeter wave flat panel device 2 and the active millimeter wave scanning assembly 1, allowing the system to enter a state to be scanned, and automatically executing a checking and spatial calibration procedure so as to ensure scanning accuracy;
S2, allowing the user to stand in a designated position according to the foot-type standing position mark on the pressure sensing device 3;
S3, measuring, via the pressure sensing device 3, pressure distribution data when the human body stands, and sending the collected pressure distribution data to the terminal control device 6;
S4, evaluating, via the terminal control device 6, whether the standing posture and standing position of the human body are standard based on the pressure distribution data, generating corresponding feedback information when the standing posture and standing position are not standard, and instructing the user to adjust the standing posture and standing position via the plantar indication module 4;
S5, repeating S3-S4 until the standing posture and standing position meet the standard requirements;
S6, starting, via the active millimeter wave flat panel device 2 and the active millimeter wave scanning assembly 1, to perform non-invasive scanning on the back of the human body; transmitting, via the active millimeter wave scanning assembly 1, a millimeter wave signal of a specific frequency band to the back of the human body, and sending the received echo signal of the back of the human body to the terminal control device 6;
S7, detecting, via the terminal control device 6, whether there is scoliosis in the human body based on the echo signal, generating a corresponding diagnosis report according to the scoliosis recognition result, and sending the scoliosis recognition result and the diagnosis report together to the terminal display device 5; and
S8, displaying, via the terminal display device 5, the scoliosis recognition result and the diagnosis report in real time, and performing, via the terminal control device 6, electronic transmission or printing for the medical personnel to further analyze and formulate the treatment plan.
\textcircled{1} In S4, evaluating, via the terminal control device 6, whether the standing posture and standing position of the human body are standard based on the pressure distribution data, generating corresponding feedback information when the standing posture and standing position are not standard, and instructing the user to adjust the standing posture and standing position via the plantar indication module 4, includes:
S41, performing digital processing on the received pressure distribution data, filtering noise, and converting the same into a data format suitable for algorithm analysis through normalization processing;
S42, performing a two-foot pressure distribution analysis based on the pre-processed pressure distribution data to determine the center of gravity weight of the human body and the pressure distribution symmetry;
S43, detecting the positions of the heel, the arch, and the forefoot of the human body based on the pre-processed pressure distribution data, and performing standing position comparison and analysis with a foot-type standing position mark on the pressure sensing device 3 (as shown in FIG. 2); and
S44, evaluating whether the standing posture and standing position of the human body are standard by combining the pressure distribution symmetry and the standing position comparison and analysis result, generating corresponding feedback information when the standing posture and standing position are not standard, and instructing the user to adjust the standing posture and standing position by the plantar indication module 4 by sending the feedback information to the plantar indication module 4;
wherein the plantar indication module 4 is configured to instruct the user to adjust the standing posture and standing position through a voice or visual prompt.
\textcircled{2} In S7, detecting, via the terminal control device 6, whether there is scoliosis in the human body based on the echo signal, generating a corresponding diagnosis report according to the scoliosis recognition result, and sending the scoliosis recognition result and the diagnosis report together to the terminal display device 5, includes:
S71, filtering and denoising the received echo signal, and converting the same into 3D morphological data of the back of the human body;
S72, recognizing the back key points from the 3D morphological data of the back of the human body using the depth learning algorithm BlazePose, and then segmenting the 3D data of the back of the human body;
S73, based on the 3D data of the back of the human body, recognizing a preliminary curve of a spine contour using an improved active contour algorithm, and then performing a cubic B-spline curve smoothing to obtain a fitting curve of the spine contour;
S74, detecting whether there is scoliosis in the human body based on the calculated Cobb angle of the spine, generating a corresponding diagnosis report according to the scoliosis recognition result, and sending the scoliosis recognition result and the diagnosis report together to the terminal display device 5;
wherein, the back key points include the bilateral acromion, scapula, back dimples, and iliac crest, and the diagnosis report includes the Cobb angle of the spine, the severity range of scoliosis, and the corresponding medical suggestions.

Specifically, in S73, based on the 3D data of the back of the human body, using an improved active contour algorithm to identify a preliminary curve of the spine contour, and then performing cubic B-spline curve smoothing to obtain a fitting curve of the spine contour, includes:
S731, initializing a curve at a central axis position of the 3D data on the back of the human body as an initial curve approximating a real spine contour;
S732, constructing by using the active contour algorithm an energy function including an internal energy term and an external energy term, wherein the internal energy maintains the smoothness and structure of the curve, and the external energy attracts the initial curve to the edge of the spine by using the gradient size based on the image gradient information;
S733, updating the initial curve position according to the gradient descending direction of the energy function, and iteratively minimizing the energy function so that the initial curve gradually approaches the real spine contour;
S734, setting a stop condition that the reduction amplitude of the energy function is lower than a threshold value or the number of iterations reaches an upper limit, and after the iteration is completed, eliminating abnormal points to obtain the preliminary curve of the spine contour; and
S735, obtaining the fitting curve of the spine contour using the cubic B-spline curve smoothing.

In the technical solution of the present application, provided is a non-radiation, high-accuracy, high-throughput, rapid periodicity, and user-friendly scoliosis recognition system and method by introducing the active millimeter wave imaging technology. Based on the millimeter wave radar principle, 3D morphological data of the back of the human body is obtained by transmitting and receiving millimeter wave signals, and then the depth learning algorithm and surface fitting algorithm are applied to respectively identify the key points of the back and analyze the 3D morphology of the spine. Finally, the Cobb angle of the spine is calculated and the corresponding diagnosis report is generated. This method not only improves the accuracy and efficiency of scoliosis recognition but also helps to reduce the risk of missed diagnosis and misdiagnosis. Meanwhile, it protects the privacy of users and lays a solid foundation for the early treatment of scoliosis patients and the improvement of quality of life.

The embodiments above are only used to illustrate the technical solution of the present invention and not to limit it. Although the present invention has been described in detail with reference to the aforementioned various embodiments, ordinary technical personnel in the art should understand that they can still modify the technical solutions recorded in the aforementioned embodiments, or equivalently replace some technical features. These modifications or substitutions do not separate the essence of the corresponding technical solutions from the spirit and scope of the technical solutions of the various embodiments of the present invention.

What is claimed is:

1. A scoliosis recognition system based on active millimeter wave imaging technology, comprising an active millimeter wave scanning assembly (1), an active millimeter wave flat panel device (2), a pressure sensing device (3), a plantar indication module (4), a terminal display device (5), and a terminal control device (6); wherein
the active millimeter wave scanning assembly (1) is configured to transmit a millimeter wave signal of a specific frequency band to the back of a human body, receive an echo signal of the back of the human body, and send the received echo signal to the terminal control device (6) so as to realize non-invasive scanning on the back of the human body;
the active millimeter wave flat panel device (2) is configured to, under the control of the terminal control device (6), drive the active millimeter wave scanning assembly (1) to scan in a sequence from top to bottom so as to obtain 3D morphological data of the back of the human body;
the pressure sensing device (3) is configured to measure pressure distribution data when the human body stands, and send the collected pressure distribution data to the terminal control device (6);
the terminal control device (6) is configured to evaluate whether the standing posture and standing position of the human body are standard based on the pressure distribution data, generate corresponding feedback information when the standing posture and standing position are not standard, and instruct the user to adjust the standing posture and standing position via the plantar indication module (4), and at the same time, detect whether there is scoliosis based on the echo signal, generate a corresponding diagnosis report according to the scoliosis recognition result, and send the scoliosis recognition result and the diagnosis report together to the terminal display device (5); and
the terminal display device (5) is configured to display the scan progress, device status, scoliosis recognition result, and diagnosis report in real time.

2. The scoliosis recognition system based on active millimeter wave imaging technology of claim 1, wherein the terminal control device (6) comprises a pressure distribution data pre-processing module, a pressure distribution data analysis module, a key position detection module, and a standing posture and standing position evaluation module; wherein
the pressure distribution data pre-processing module is configured to perform digital processing on the received pressure distribution data, filter noise, and convert the same into a data format suitable for algorithm analysis through normalization processing;
the pressure distribution data analysis module is configured to perform a two-foot pressure distribution analysis based on the pre-processed pressure distribution data, and determine the center of gravity weight of the human body and the pressure distribution symmetry;
the key position detection module is configured to detect the positions of the heel, the arch, and the forefoot of the human body based on the pre-processed pressure distribution data, and perform standing position comparison and analysis with a foot-type standing position mark on the pressure sensing device (3);
the standing posture and standing position evaluation module is configured to evaluate whether the standing posture and standing position of the human body are standard by combining the pressure distribution symmetry and the standing position comparison and analysis result, generate corresponding feedback information when the standing posture and standing position are not standard, and instruct the user to adjust the standing posture and standing position by the plantar indication module (4) by sending the feedback information to the plantar indication module (4);
wherein the plantar indication module (4) is configured to instruct the user to adjust the standing posture and standing position through a voice or visual prompt.

3. The scoliosis recognition system based on active millimeter wave imaging technology of claim 2, wherein the pressure distribution data analysis module being configured to perform a two-foot pressure distribution analysis based on the pre-processed pressure distribution data to determine the center of gravity weight of the human body and the pressure distribution symmetry, comprises:
calculating the two-foot pressure distribution using the following formula:
$$\begin{matrix}{C}_{L} ( t ) = \frac{{{\sum }^{m}}_{i = 1} [ {P}_{i} ( t ) {x}_{i} ]}{{{\sum }^{m}}_{i = 1} {P}_{i} ( t )} ; \\ {C}_{R} ( t ) = \frac{{{\sum }^{n}}_{j = 1} [ {P}_{j} ( t ) {y}_{j} ]}{{{\sum }^{n}}_{j = 1} {P}_{j} ( t )} ;\end{matrix}$$
where C_{L}(t) is the left foot pressure value distribution at time t; Pᵢ(t) is the pressure value detected by the ith pressure sensor below the left foot at time t; xᵢ is the coordinate of the ith pressure sensor below the left foot on the plane; and m is the number of pressure sensors below the left foot;
C_{R}(t) is the pressure value distribution of the right foot at time t; Pⱼ(t) is the pressure value detected by the jth pressure sensor below the right foot at time t; yⱼ is the coordinate of the jth pressure sensor below the right foot on the plane; and n is the number of pressure sensors below the right foot;
calculating the center of gravity weight of the human body based on the two-foot pressure distribution using the following formula:
$$\begin{matrix}{W}_{L} ( t ) = \frac{{C}_{L} ( t ) {S}_{L} + {C}_{R} ( t ) {S}_{L}}{{S}_{L} + {S}_{R}} ; \\ {W}_{R} ( t ) = \frac{{C}_{L} ( t ) {S}_{R} + {C}_{R} ( t ) {S}_{R}}{{S}_{L} + {S}_{R}} ;\end{matrix}$$
where W_{L}(t) and W_{R}(t) are respectively the center of gravity weight of the left foot and the center of gravity weight of the right foot at time t; and S_{L} and S_{R} are respectively the contact area of the left foot and the contact area of the right foot;
based on the center of gravity weight of the human body, calculating a pressure distribution symmetry score using the following formula:
$$S ( t ) = 1 - \frac{\vert {W}_{L} ( t ) - {W}_{R} ( t ) \vert}{max [ {W}_{L} ( t ) , {W}_{R} ( t ) ]} ;$$
where S(t) is the pressure distribution symmetry score at time t, and when the pressure distribution symmetry score is higher than 0.85, it indicates that the standing posture of the human body is symmetrical and meets the requirements of posture symmetry; otherwise, the standing posture of the human body is asymmetrical and does not meet the requirements of posture symmetry.

4. The scoliosis recognition system based on active millimeter wave imaging technology of claim 1, wherein the terminal control device (6) comprises an echo signal pre-processing module, a back key point recognition module, a spine fitting analysis module, and a diagnosis report generation module; wherein
the echo signal pre-processing module is configured to filter and denoise the received echo signal, and convert the same into 3D morphological data of the back of the human body;
the back key point recognition module is configured to recognize the back key points from the 3D morphological data of the back of the human body using the depth learning algorithm BlazePose, and then segment the 3D data of the back of the human body;
the spine fitting analysis module is configured to, based on the 3D data of the back of the human body, recognize a preliminary curve of a spine contour using an improved active contour algorithm, and then perform a cubic B-spline curve smoothing to obtain a fitting curve of the spine contour;
the diagnosis report generation module is configured to detect whether there is scoliosis in the human body based on the calculated Cobb angle of the spine, generate a corresponding diagnosis report according to the scoliosis recognition result, and send the scoliosis recognition result and the diagnosis report together to the terminal display device (5);
wherein, the back key points comprise the bilateral acromion, scapula, back dimples, and iliac crest, and the diagnosis report comprises the Cobb angle of the spine, the severity range of scoliosis, and the corresponding medical suggestions.

5. A scoliosis recognition method based on active millimeter wave imaging technology, which is applied to the scoliosis recognition system based on active millimeter wave imaging technology of claim 1, comprising the steps of:
S1, starting the active millimeter wave flat panel device (2) and the active millimeter wave scanning assembly (1), allowing the system to enter a state to be scanned, and automatically executing a checking and spatial calibration procedure so as to ensure scanning accuracy;
S2, allowing the user to stand in a designated position according to the foot-type standing position mark on the pressure sensing device (3);
S3, measuring, via the pressure sensing device (3), pressure distribution data when the human body stands, and sending the collected pressure distribution data to the terminal control device (6);
S4, evaluating, via the terminal control device (6), whether the standing posture and standing position of the human body are standard based on the pressure distribution data, generating corresponding feedback information when the standing posture and standing position are not standard, and instructing the user to adjust the standing posture and standing position via the plantar indication module (4);
S5, repeating S3-S4 until the standing posture and standing position meet the standard requirements;
S6, starting, via the active millimeter wave flat panel device (2) and the active millimeter wave scanning assembly (1), to perform non-invasive scanning on the back of the human body; transmitting, via the active millimeter wave scanning assembly (1), a millimeter wave signal of a specific frequency band to the back of the human body, and sending the received echo signal of the back of the human body to the terminal control device (6);
S7, detecting, via the terminal control device (6), whether there is scoliosis in the human body based on the echo signal, generating a corresponding diagnosis report according to the scoliosis recognition result, and sending the scoliosis recognition result and the diagnosis report together to the terminal display device (5); and
S8, displaying, via the terminal display device (5), the scoliosis recognition result and the diagnosis report in real time, and performing, via the terminal control device (6), electronic transmission or printing for the medical personnel to further analyze and formulate the treatment plan.

6. The scoliosis recognition method based on active millimeter wave imaging technology of claim 5, wherein: in S4, evaluating, via the terminal control device (6), whether the standing posture and standing position of the human body are standard based on the pressure distribution data, generating corresponding feedback information when the standing posture and standing position are not standard, and instructing the user to adjust the standing posture and standing position via the plantar indication module (4), comprises:
S41, performing digital processing on the received pressure distribution data, filtering noise, and converting the same into a data format suitable for algorithm analysis through normalization processing;
S42, performing a two-foot pressure distribution analysis based on the pre-processed pressure distribution data to determine the center of gravity weight of the human body and the pressure distribution symmetry;
S43, detecting the positions of the heel, the arch, and the forefoot of the human body based on the pre-processed pressure distribution data, and performing standing position comparison and analysis with a foot-type standing position mark on the pressure sensing device (3); and
S44, evaluating whether the standing posture and standing position of the human body are standard by combining the pressure distribution symmetry and the standing position comparison and analysis result, generating corresponding feedback information when the standing posture and standing position are not standard, and instructing the user to adjust the standing posture and standing position by the plantar indication module (4) by sending the feedback information to the plantar indication module (4);
wherein the plantar indication module (4) is configured to instruct the user to adjust the standing posture and standing position through a voice or visual prompt.

7. The scoliosis recognition method based on active millimeter wave imaging technology of claim 6, wherein in S7, detecting, via the terminal control device (6), whether there is scoliosis in the human body based on the echo signal, generating a corresponding diagnosis report according to the scoliosis recognition result, and sending the scoliosis recognition result and the diagnosis report together to the terminal display device (5), comprises:
S71, filtering and denoising the received echo signal, and converting the same into 3D morphological data of the back of the human body;
S72, recognizing the back key points from the 3D morphological data of the back of the human body using the depth learning algorithm BlazePose, and then segmenting the 3D data of the back of the human body;
S73, based on the 3D data of the back of the human body, recognizing a preliminary curve of a spine contour using an improved active contour algorithm, and then performing a cubic B-spline curve smoothing to obtain a fitting curve of the spine contour;
S74, detecting whether there is scoliosis in the human body based on the calculated Cobb angle of the spine, generating a corresponding diagnosis report according to the scoliosis recognition result, and sending the scoliosis recognition result and the diagnosis report together to the terminal display device (5);
wherein, the back key points comprise the bilateral acromion, scapula, back dimples, and iliac crest, and the diagnosis report comprises the Cobb angle of the spine, the severity range of scoliosis, and the corresponding medical suggestions.

8. The scoliosis recognition method based on active millimeter wave imaging technology of claim 7, wherein in S73, based on the 3D data of the back of the human body, recognizing a preliminary curve of a spine contour using an improved active contour algorithm, and then performing a cubic B-spline curve smoothing to obtain a fitting curve of the spine contour, comprises:
S731, initializing a curve at a central axis position of the 3D data on the back of the human body as an initial curve approximating a real spine contour;
S732, constructing by using the active contour algorithm an energy function including an internal energy term and an external energy term, wherein the internal energy maintains the smoothness and structure of the curve, and the external energy attracts the initial curve to the edge of the spine by using the gradient size based on the image gradient information;
S733, updating the initial curve position according to the gradient descending direction of the energy function, and iteratively minimizing the energy function so that the initial curve gradually approaches the real spine contour;
S734, setting a stop condition that the reduction amplitude of the energy function is lower than a threshold value or the number of iterations reaches an upper limit, and after the iteration is completed, eliminating abnormal points to obtain the preliminary curve of the spine contour; and
S735, obtaining the fitting curve of the spine contour using the cubic B-spline curve smoothing.
